(19) United States
(12) Patent Application Publication
(10) Pub. No.: US 2016/0262842 A1
(43) Pub. Date: Sep. 15, 2016

(54) MODULAR SURGICAL ACCESSORY TABLE

(71) Applicant: VARIAMED LLC, Chattanooga, TN (US)

(72) Inventors: David SELLERS, Chattanooga, TN (US); Kevin PAGE, Chattanooga, TN (US); David KING, Chattanooga, TN (US)

(21) Appl. No.: 15/065,172

(22) Filed: Mar. 9, 2016

Related U.S. Application Data
(60) Provisional application No. 62/130,055, filed on Mar. 9, 2015.

(51) Int. Cl. A61B 50/15; A47B 47/00; F16B 12/10; A61B 50/13

(52) U.S. Cl. CPC A61B 50/15; A61B 50/13; A47B 47/0091; F16B 12/10

(57) ABSTRACT

A modular table assembly for an operating room includes an arcuate corner module and at least one rectangular side module. The modules, which are interconnected by pins that can be released by depressing a pedal, are easy to separate, move and reassemble. The modular design enables one to reconfigure the table layout according to the geometry of the room.

### This application claims benefit from provisional application 62/130055, filed Mar. 9, 2015.

### BACKGROUND OF THE INVENTION

This invention relates to an accessory table for use in an operating room.

Conventional surgical accessory tables have flat, featureless horizontal tops, and do not make best use of corner space. A standard table is of fixed size and cannot be easily altered in shape according to its intended environment, and can be difficult to move about especially when items have been placed on the table.

### SUMMARY OF THE INVENTION

An object of the invention is to provide a surgical accessory table composed of modules which are easily transported, interconnected and draped.

Another object is to provide a table which makes better use of space at a corner of a room.

A further object is to reduce the total cost of back table(s) and draping in an operating room.

These and other objects are attained by a surgical accessory table as described below.

### BRIEF DESCRIPTION OF THE DRAWINGS

In the accompanying drawings,

FIG. 1 is a perspective view of a surgical accessory table embodying the invention from above and slightly to the rear;

FIG. 2 is a perspective view thereof, from above and one side;

FIG. 3 is a perspective view thereof, from above and the front;

FIG. 4 is a top plan view thereof;

FIG. 5 is a front elevational view thereof;

FIGS. 6a6fare views of a side table component of the invention, showing preferred dimensions;

FIGS. 7a -7eare view of a corner table component of the invention, showing preferred dimensions;

FIGS. 8a-8cshow details of a releasable pin connection between the corner table and one of the side tables;

FIGS. 9a-9c are top plan views showing the releasable pin connection; and

FIGS. 10a-10e are detail views of a side bowl support attachment for the side tables.

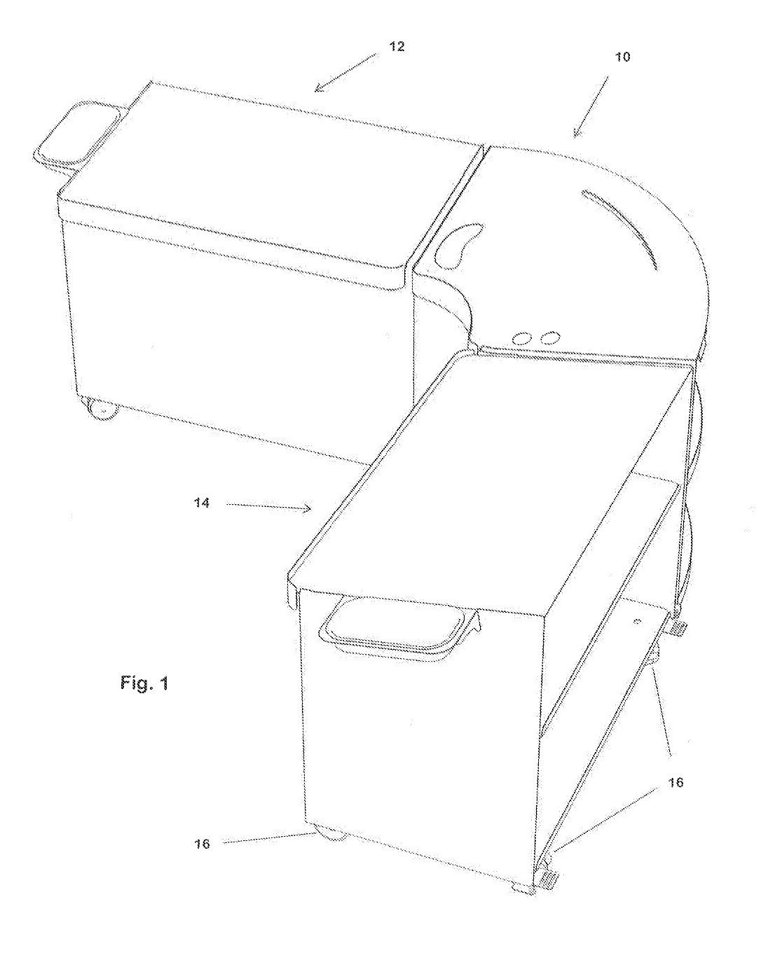

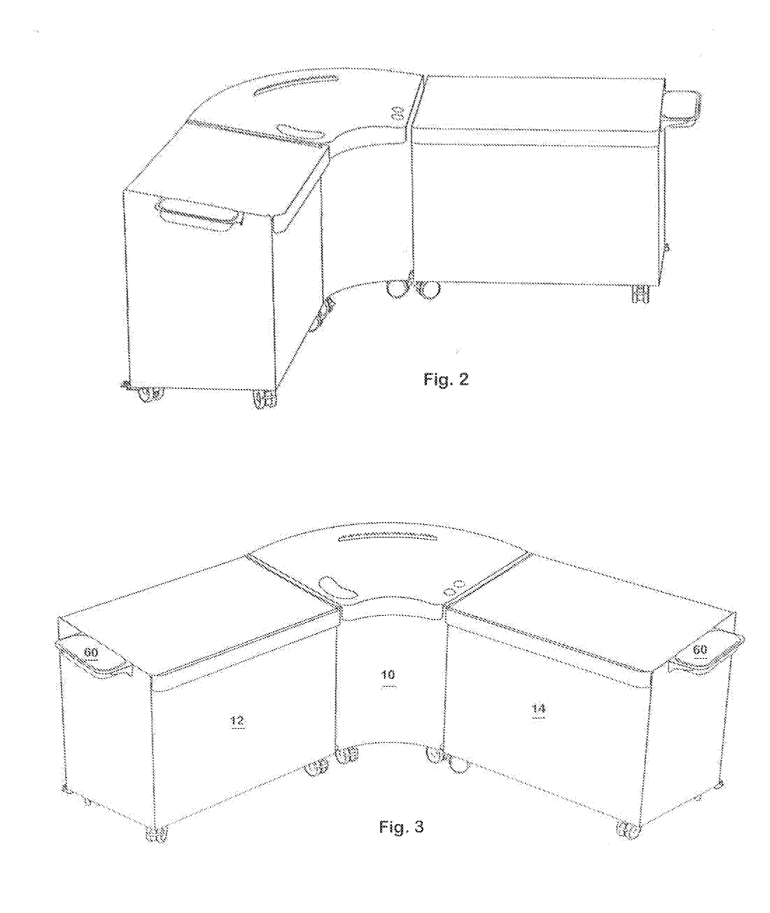

### DESCRIPTION OF THE PREFERRED EMBODIMENT

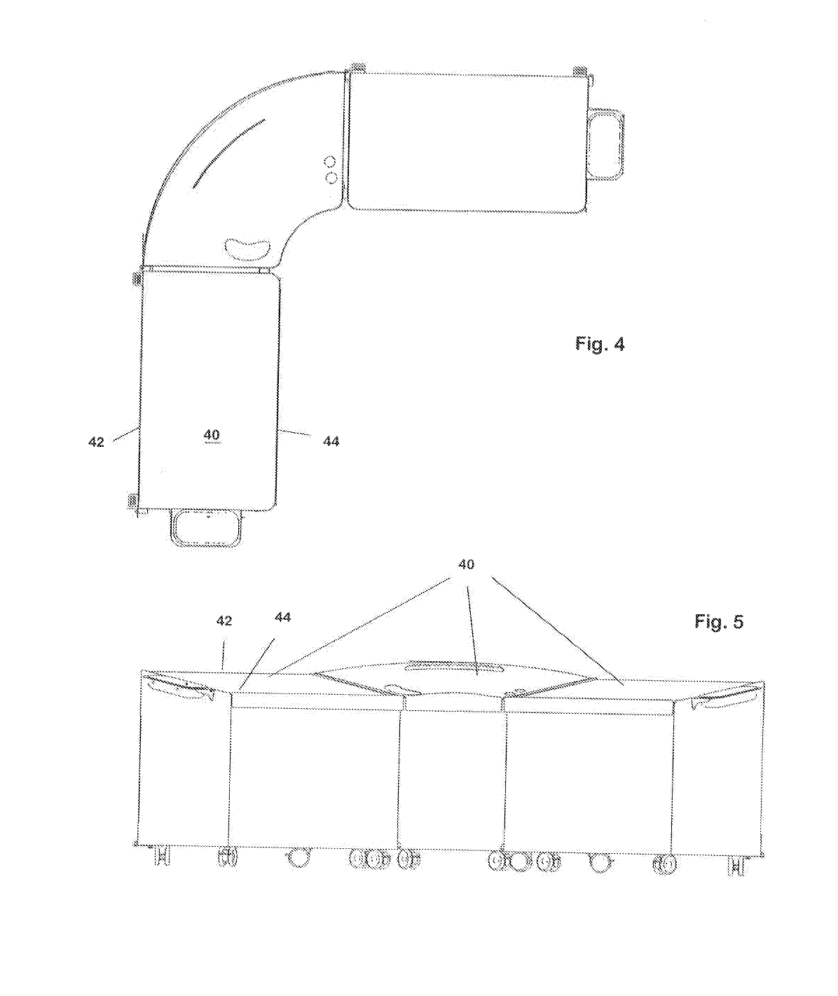

A surgical accessory table embodying the invention includes three modular table sections, including a corner module 10 and two identical side modules 12, 14, which can be interconnected as desired to form a versatile back table assembly. The side modules 12, 14 are rectangular in plan view (FIG. 4), whereas the plan view of the corner module is an annular arc of about 90′. The sides 18, 19 of the corner module lie in vertical planes which are perpendicular to one another.

Each module has wheels or casters 16 as shown.

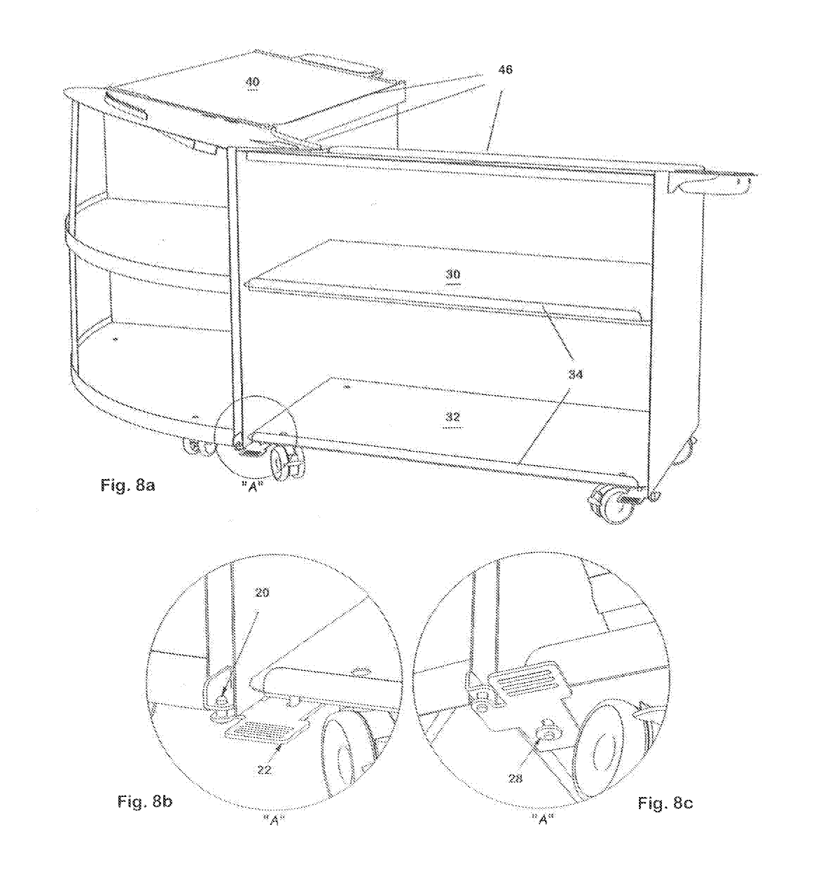

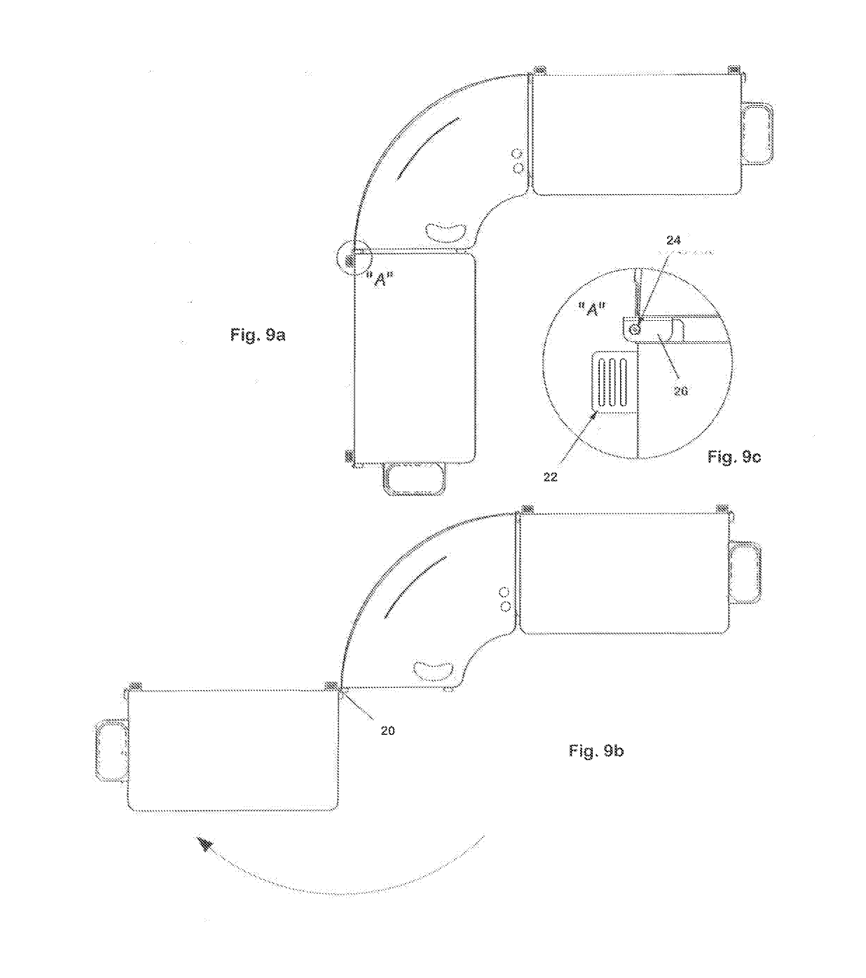

Details of the interconnecting structure are shown in FIGS. 8b, and 9e. Each side module has, at one lower rear corner thereof, a vertically movable pin 20 (FIG. 8b) which is connect to and biased upward by a resilient pedal 22. One end of the pedal is rigidly attached (e.g., by welding) to the side module's structure, so that the pedal acts as a leaf spring.

The forward end of the pedal protrudes horizontally from a lower corner of the side module. In its normal horizontal position, the pin 20 seats in a hole 24. (FIG. 9c) in receptacle 26 attached to a neighboring module. One can release the pin from the hole by depressing the pedal. A pedal stop 28 (FIG. 8c) prevents overtravel.

As FIG. 9b shows, the pin 20, when engaged, serves as a hinge pin, allowing one module to be pivoted away from its neighbor without becoming disconnect from it.

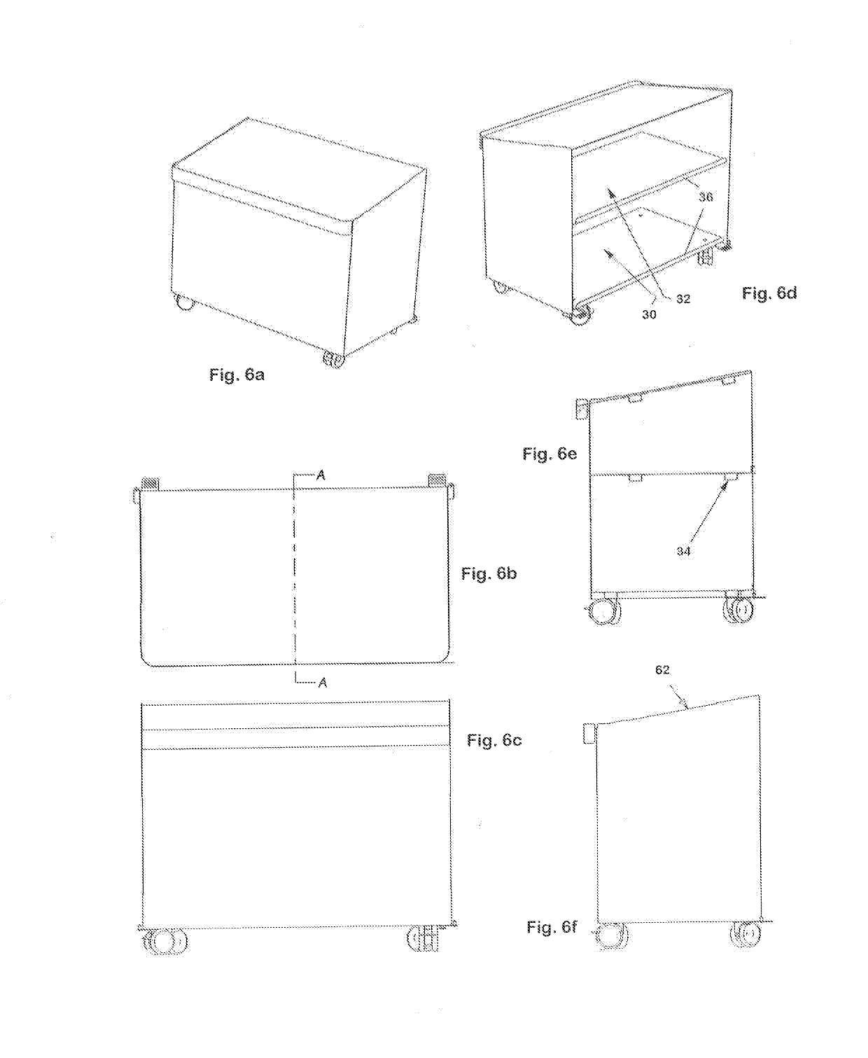

As best seen in FIG. 8a, each of the modules 10, 12, 14 has two horizontal shelves 30, 32, accessible from the rear. The shelves are reinforced by braces 32 (FIG. 6e. Each shelf has a short vertical lip 36 at its exposed (rearward) edge to prevent items from falling off. The shelves provide the functionality of a separate open case cart.

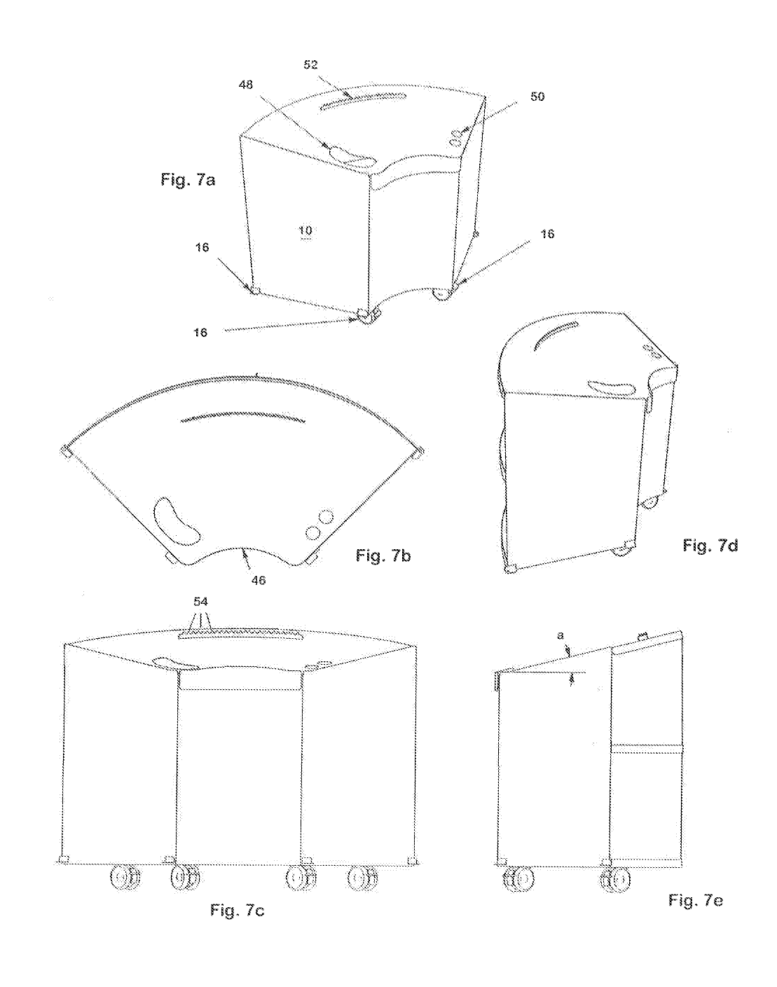

As illustrated in FIG. 5, the top surface 40 of each module is not horizontal, but rather is inclined downward in the direction of the user, from its rear edge 42 to its front edge 44, at an angle “a” (FIG. 7e) of about 10°-14′. The inclination makes items toward the rear of the table easier to reach. A lip 46 (best seen in FIG. 8a) at the forward (lower) edge of each top surface keeps items from rolling off the inclined surface.

As shown in FIG. 7a, the corner module 10 has a kidney-shaped aperture 48 or depression at one side thereof, for receiving a standard “kidney” bowl (not shown), and a pair of circular apertures 50 or depressions, for receiving specimen cups (not shown).

Toward the rear of the corner module's top, there is an integral instrument holder 52, which has upwardly extending rounded teeth 54 (FIG. 7c) for holding instruments securely. Providing an instrument holder integral with the table top avoids the need to roll towels to retain instruments placed on the table.

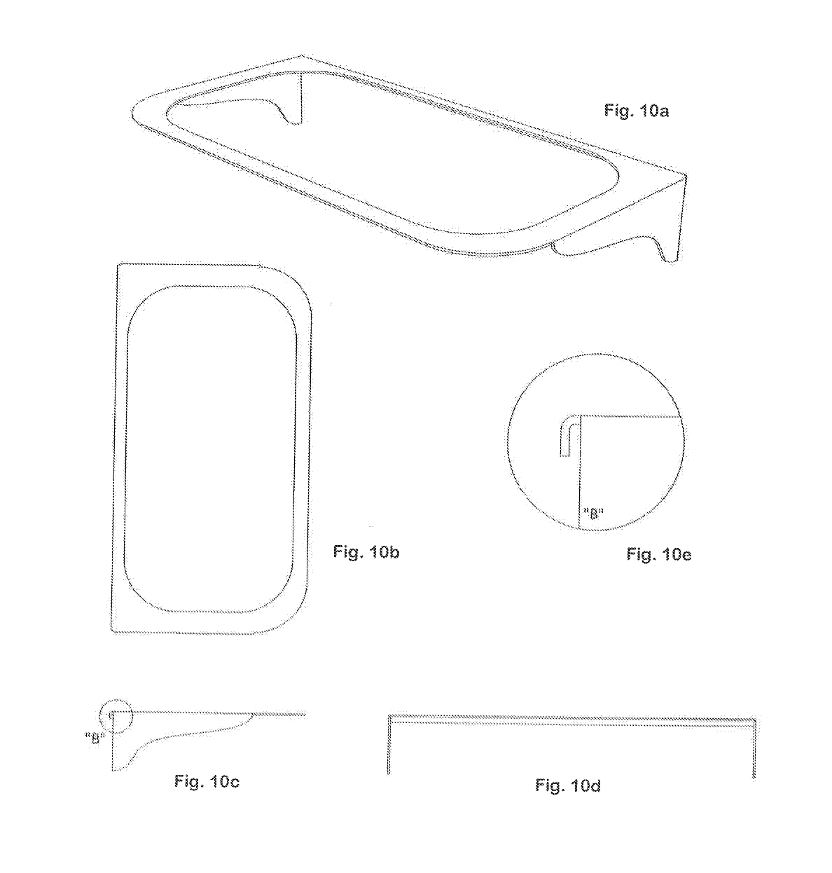

The side bowl supports 60 seen at either end of the assembly (e.g., in FIG. 3) are shown in detail in FIGS. 8a-8d. As indicated in FIG. 6f, each side of each side module is notched to receive a hook 62 (FIG. 10e) on a respective side bowl support.

The invention, particularly when used with draping made specifically for the table, provides several advantages over conventional tables, as shown in the following table:

| Feature | Standard Table | Present Invention |
|---|---|---|
| Overall lengths (ft.) | 5-7 | 9 |
| Surface area (sq. in.) | 2520 | 4380 |
| Surface inclination (deg.) | 0 | 10-14 |
| Modular? | No | Yes |
| Kidney basin holder? | No | Yes |
| End basin holder? | No | Yes |
| Specimen cup holder? | No | Yes |
| Instrument holder? | No | Yes |
| Time to drape table | 8 min. | 30-45 sec. |

The shapes of the modules shown in the drawings are those presently preferred. It should be understood, however, that the invention is not limited to modules of particular sizes or proportions, and that it is expected the invention may be made in other shapes.

And while two side modules are shown in the drawings and described above, this invention could be practiced just as well with only one side module, in addition to the corner module. Conversely, more modules than those shown could be interconnected.

Inasmuch as the invention is subject to variations and modifications, it should be understood that the foregoing is merely illustrative of the invention, which is to be measured by the claims below.

What is claimed is:

1. A modular table assembly for an operating room, said assembly comprising
a corner module having, in plan view, an arcuate shape, and
at least one side module having, in plan view, a rectangular shape,
said modules having releasable connectors so that they can he assembled in an operating room.

2. The invention of claim 1, wherein each of said modules has a top surface which is inclined from horizontal from a lower edge at a front side of the module to a higher edge at a rear side of the module.

3. The invention of claim 1, wherein said corner module has vertical end surfaces lying in planes perpendicular to one another.

4. The invention of claim 1, wherein each of said modules has a lip at its lower forward edge to retain instruments placed on top of the module.

5. The invention of claim 1, wherein at least one of said modules has a kidney-shaped recess or opening to receive a kidney basin.

6. The invention of claim 1, wherein at least one of said modules has plural openings in its top surface for holding specimen cups.

6. The invention of claim 1, wherein at least one of said modules has, formed on its top surface, an element having plural upwardly extending rounded teeth for holding instruments.

7. The invention of claim 1 wherein each of said modules has plural shelves below its top surface.

8. The invention of claim 1, wherein each of said connectors comprises a pin on its respective module, adapted to he received in an aperture in an adjoining module, and further comprising a release pedal connected to the pin so that the pin can be disengaged from the aperture by pressing on the pedal.

9. The invention of claim 8, wherein the pedal is flexible, having one end rigidly affixed to a base of its module, so that the pedal acts as a leaf spring urging the pin to its upward engaged position.

10. The invention of claim 1, comprising at least two of said side modules.
